(19) United States
(12) Patent Application Publication
(10) Pub. No.: US 2019/0330832 A1
(43) Pub. Date: Oct. 31, 2019

(54) DEODORIZER

(71) Applicant: LIXIL Corporation, Tokyo (JP)

(72) Inventors: TingJian GONG, Tokyo (JP); Toshihiko KOMATSU, Tokyo (JP); Masashi KONNO, Tokyo (JP); Motoki KAGAWA, Tokyo (JP); Masato WAKABAYASHI, Tokyo (JP); Tomoya SASAKI, Tokyo (JP); YinTao LIU, Tokyo (JP); Atsushi WAKAMATSU, Tokyo (JP)

(73) Assignee: LIXIL Corporation, Tokyo (JP)

(21) Appl. No.: 16/464,251

(22) PCT Filed: Nov. 7, 2017
(86) PCT No.: PCT/JP2017/040111

§ 371 Date: May 24, 2019

(30) Foreign Application Priority Data
Nov. 29, 2016 (JP) 2016-231305
Nov. 29, 2016 (JP) 2016-231306
Nov. 29, 2016 (JP) 2016-231307

(51) Int. Cl. E03D 9/052; A61L 9/00

(52) U.S. Cl. CPC E03D 9/052; A61L 9/00

(57) ABSTRACT

The purpose of the present invention is to provide a deodorizer in which spray, etc. can be prevented or inhibited from entering the interior. A private part washing device 3 functioning as a deodorizer device, provided with: a case 7 provided to the rear part of a toilet bowl 4 provided to a main toilet body 2, the case having an opening part 9 opening onto the toilet bowl 4; an air duct 24 having a blowout port 28 from which airflow is blown out, the blowout port 28 opening from the opening part 9 to the outside of the case 7; and a shutter 10 for opening/closing the opening part 9, the shutter 10 forming an air outlet 38 between the shutter 10 and the case 7 when the opening part 9 is closed. When the opening part 9 is closed by the shutter 10, airflow is guided in a first direction from the air outlet 38 into the toilet bowl 4, and when the opening part 9 is opened by the shutter 10, airflow is guided in a direction different from the first direction.

### TECHNICAL FIELD

The present invention relates to a deodorizer for a toilet.

### BACKGROUND ART

Conventionally, in a deodorizer for a toilet, a blowout port, from which airflow is blown out, is provided on an inner side of a shutter that pivots vertically (for example, refer to Patent Document 1). In the deodorizer, during deodorization, the shutter is pivoted upward to come into a downward opened state in which the shutter is opened from below and the airflow blown out from the blowout port is guided to an inner surface of the toilet bowl. On the other hand, during drying of an anus, the shutter is pivoted downward to come into an upward opened state in which the shutter is opened from above, and the airflow blown out from the blowout port is guided to the anus. In addition, in the deodorizer for a toilet described in Patent Document 1, air containing odor is suctioned by a blower and is caused to pass through a deodorizing cartridge, and thereby deodorization is performed.
Patent Document 1: Japanese Unexamined Patent Application, Publication No. 2015-206184

### DISCLOSURE OF THE INVENTION

### Problems to be Solved by the Invention

In such a deodorizer, a reduction in operation of the shutter needs to result in preventing or inhibiting spray, etc. from entering the interior. In addition, in recent years, from a viewpoint of securing an installation space with consideration for high functionality of a washing device that is provided in a toilet, there is a demand for a compact deodorizer that maintains a deodorizing function as is.

The invention is made with consideration for the above description, and an object thereof is to provide a deodorizer in which spray, etc. can be prevented or inhibited from entering the interior. In addition, the invention is made to provide a deodorizer that can be compact while maintaining a deodorizing function as is.

### Means for Solving the Problems

In order to achieve the object described above, the invention provides a deodorizer (private part washing device 3) including: a case (for example, a case 7 to be described below) provided to the rear part of a toilet bowl (for example, a toilet bowl 4 to be described below) provided to a main toilet body (for example, a main toilet body 2 to be described below), the case having an opening part (for example, an opening part 9 to be described below) opening onto the toilet bowl; an air duct (for example, an air duct 24 to be described below) having a blowout port (for example, a blowout port 28 to be described below) from which airflow is blown out, the blowout port opening from the opening part to the outside of the case; and a shutter (for example, a shutter 10 to be described below) for opening/closing the opening part, the shutter forming an air outlet (for example, an air outlet 38 to be described below) between the shutter and an edge of the opening part when the opening part is closed. When the opening part is closed by the shutter, the airflow is guided in a first direction from the air outlet into the toilet bowl, and when the opening part is opened by the shutter, the airflow is guided in a direction different from the first direction.

It is preferable that the first direction be a direction along an inner surface of the toilet bowl.

It is preferable that the case have a rib (for example, a rib 37 to be described below) provided along an edge of the opening part, the rib guiding the airflow in the first direction from the air outlet when the opening part is closed by the shutter.

It is preferable that the rib be curved along the edge of the opening part.

It is preferable that a tip portion of the rib be provided below the opening part and be inclined toward a rear side of the toilet bowl as being downward in side view of the main toilet body.

It is preferable that the deodorizer further include a blocking unit (for example, a blocking plate 36 to be described below) that guides the airflow from the blowout port to the air outlet when the opening part is closed by the shutter.

It is preferable that the deodorizer further include: a first blower (for example, a first blower 25 to be described below) provided in the air duct; a deodorizing duct (for example, a deodorizing duct 29 to be described below) that has a suction port (for example, a suction port 32 to be described below) which suctions air in the toilet bowl, that deodorizes the air in the toilet bowl, the air being suctioned from the suction port, and that guides the air to the outside of the toilet bowl; a second blower (for example, a second blower 30 to be described below) provided in the deodorizing duct; and a control unit (for example, a control board 16 to be described below) that controls drive of the first blower and the second blower, in which the control unit increases an output of the second blower more than an output of the first blower when the opening part is closed by the shutter.

In addition, the invention provides a deodorizer (for example, a private part washing device 3 to be described below) including: a second blower (for example, a second blower 30 to be described below) having inlet ports (for example, inlet ports 134 and 135 to be described below) on both end portions, respectively, in an axial direction and a blowout port (for example, a blowout port 136 to be described below) on a side portion in a direction intersecting the axial direction; and a case (for example, a deodorizing duct 29 to be described below) that accommodates the second blower and forms a flow channel of air on both sides of the second blower in an axial direction, the air being suctioned from the inlet port.

It is preferable that the second blower have the inlet ports that are horizontally installed upward and downward, and the case form the flow channel above and below the second blower.

It is preferable that the case have a top plate (for example, a top plate 137 to be described below) that is extended obliquely upward from a side of the second blower to above the second blower and has a portion opposite to the inlet port of the second blower, the portion bulging upward to form the flow channel.

It is preferable that the second blower have a part that comes into contact with the top plate.

It is preferable that the deodorizer further include a packing (for example, a packing 138 to be described below) disposed along an edge of the downward inlet port, the packing being sandwiched between an inner surface of the case and an underside of the second blower, and the packing having a notch (for example, a notch 139 to be described below) that functions as the flow channel.

In addition, the invention provides a deodorizer (for example, a private part washing device 3 to be described below) including: a base (for example, a base 8 to be described below) that is disposed to the rear part of the toilet bowl (for example, a toilet bowl 4 to be described below) provided to a main toilet body (for example, a main toilet body 2 to be described below) and has a blowout port (for example, a blowout port 33 to be described below) on a side, the blowout port being opened obliquely downward; and a cover (for example, a cover 11 to be described below) that covers the base so as to form a space on the base.

It is preferable that the cover block at least a part of the blowout port in side view.

It is preferable that the base be mounted on the rear part of the toilet bowl with a gap formed between the blowout port and an upper peripheral edge of the toilet bowl.

It is preferable that the deodorizer further include a deodorizing part (for example, a deodorizing cartridge 31 to be described below) that removes odor contained in air, which passes therethrough, and releases the air, has passed therethrough, obliquely downward, and the blowout port blow out the air released from the deodorizing part, to the outside of the base.

### Effects of the Invention

According to the invention, it is possible to provide a deodorizer that is capable of preventing or inhibiting spray, etc. from entering the interior because, when an opening part opened/closed by a shutter is closed, it is possible to guide airflow from an air outlet formed between the shutter and an edge of the opening part. In addition, according to the invention, it is possible to provide a deodorizer that can be compact while maintaining a deodorizing function as is, because the deodorizer includes a blower having inlet ports on both end portions of the blower in an axial direction and a case that forms a flow channel of air on both sides of the blower in the axial direction, the air being suctioned from the inlet ports. In addition, according to the invention, it is possible to provide a deodorizer that can be compact while maintaining a deodorizing function.

### BRIEF DESCRIPTION OF THE DRAWINGS

FIG. 1 is a perspective view of a toilet including a private part washing device according to an embodiment of the invention when viewed from obliquely above on a front side.

FIG. 2 is a top view of the toilet according to the embodiment when a toilet seat, a toilet lid, and a cover of the private part washing device are detached and a shutter is closed.

FIG. 3A is a front view illustrating an opening part and a peripheral portion thereof when the shutter is detached and a second shutter is closed.

FIG. 3B is a front view illustrating the opening part and the peripheral portion thereof when the shutter is detached and the second shutter is opened.

FIG. 4A is a front view illustrating the shutter in a closed state and a peripheral portion thereof.

FIG. 4B is a side view illustrating the shutter in the closed state and the peripheral portion thereof.

FIG. 4C is a cross-sectional view of the shutter and the peripheral portion thereof when viewed in a direction of arrow IVC-IVC in FIG. 4A.

FIG. 5A is a cross-sectional view of the shutter in a downward opened state and the peripheral portion thereof.

FIG. 5B is a cross-sectional view of the shutter in an upward opened state and the peripheral portion thereof.

FIG. 6A is a cross-sectional perspective view of a deodorizing unit when viewed in a direction of arrow II-II in FIG. 2.

FIG. 6B is a cross-sectional view of the deodorizing unit when viewed in a direction of arrow in FIG. 2.

FIG. 7 is a bottom view of a second blower when a packing is bonded.

FIG. 8 is a perspective view of the private part washing device when viewed from obliquely below on the front side.

FIG. 9 is a cross-sectional view of the deodorizing unit when viewed in a direction of arrow IV-IV in FIG. 2.

### PREFERRED MODE FOR CARRYING OUT THE INVENTION

Hereinafter, an embodiment of the invention will be described in detail with reference to the drawings. Incidentally, in the following description, for convenience, a right-left direction means a right-left direction when viewed from a toilet user who sits on a toilet seat.

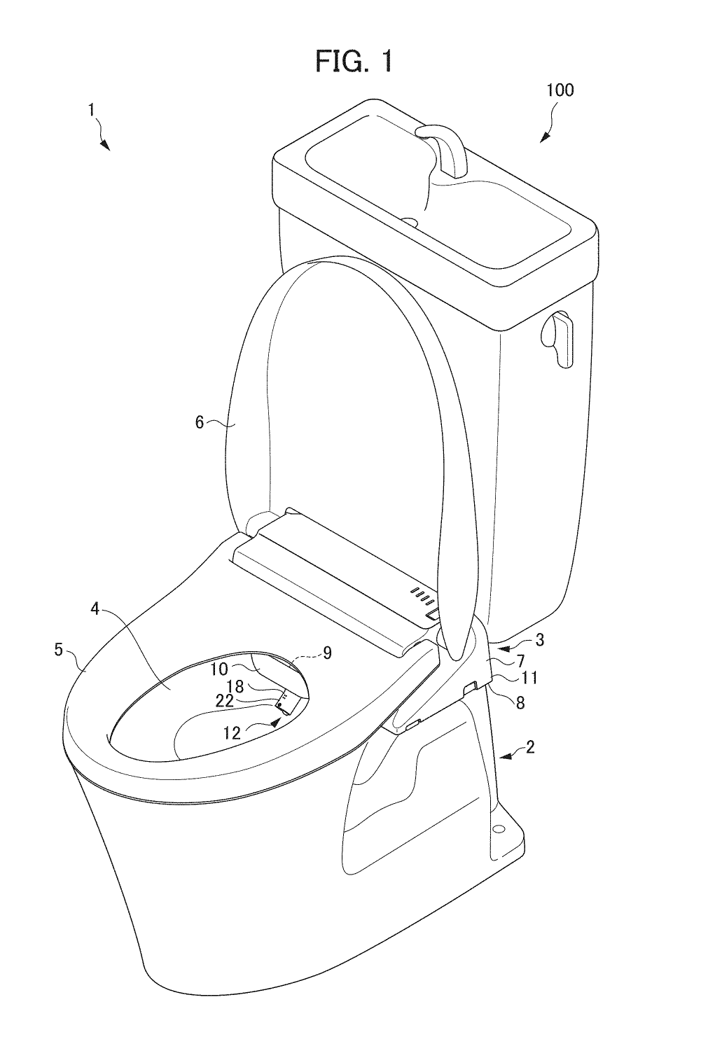

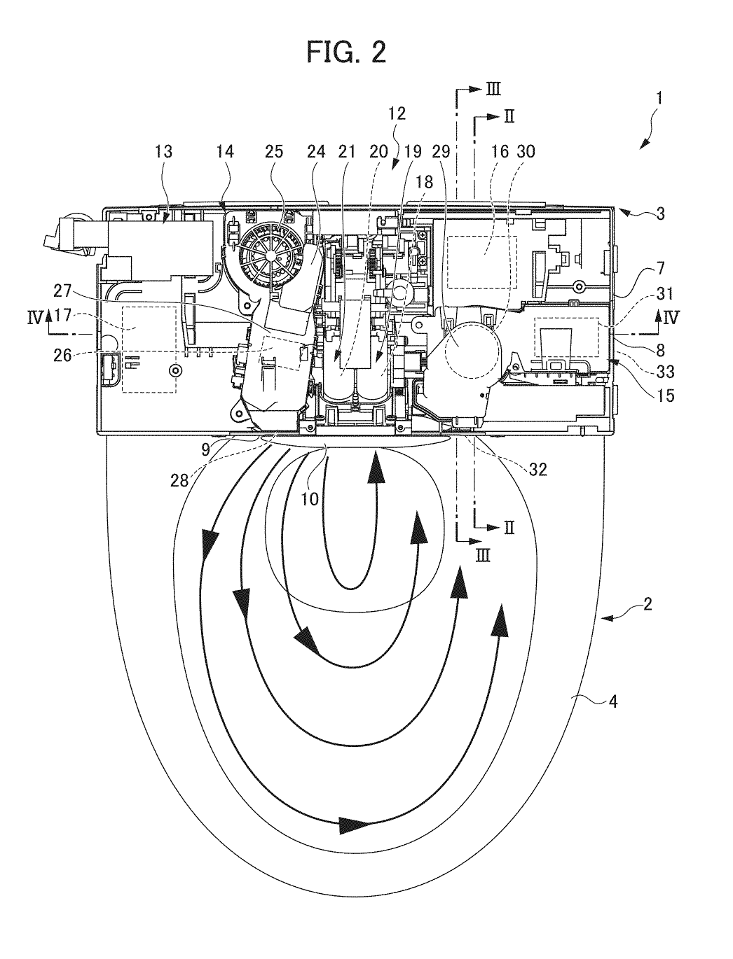

First, a toilet 1 is described with reference to FIGS. 1 and 2. FIG. 1 is a perspective view of the toilet 1 including a private part washing device 3 according to an embodiment of the invention when viewed from obliquely above on a front side. FIG. 2 is a top view of the toilet 1 when a toilet seat 5, a toilet lid 6, and a cover 11 of the private part washing device 3 are detached and a shutter 10 is closed.

The toilet 1 illustrated in FIGS. 1 and 2 is a flush toilet and includes a main toilet body 2, the private part washing device 3, and the like. In addition, the toilet 1 includes a tank 100 that stores washing water, a pump that supplies the washing water, a valve, a tube system, and the like; however, the description thereof is omitted.

The main toilet body 2 is configured to include a toilet bowl 4 that receives filth, a drain pipe (not illustrated) extended downward from a bottom portion of the toilet bowl 4, and the toilet seat 5 and the toilet lid 6 that is opened from and closed to the toilet bowl 4. In an upper portion of the toilet bowl 4, a rim flow channel (not illustrated) that generates a swirl flow of the washing water along a peripheral edge of the toilet bowl and a dropping flow channel (not illustrated) that generates a dropping flow of the swirl flow toward the drain pipe are formed.

The private part washing device 3 is provided to the rear part of the toilet bowl 4. The private part washing device 3 includes a case 7 that accommodates various functional components for private part washing, hot air drying, or the like. The case 7 includes a base 8 disposed on an upper peripheral edge of the toilet bowl 4, the shutter 10 for opening/closing a horizontally-elongated opening part 9 formed at the center on a front surface of the base 8, and the cover 11 that is assembled to the base 8. The shutter 10 and a peripheral portion thereof will be described below in detail. The toilet seat 5 and the toilet lid 6 are each pivotably attached to the cover 11.

The private part washing device 3 includes a nozzle unit 12 that discharges washing water, a warm water supply unit 13 that supplies warm water to the nozzle unit 12, an air blowing unit 14 that blows out airflow, a deodorizing unit 15 that removes odor generated from filth, a control board 16 that allows the units 12 to 15 to be driven, a power supply board 17 that supplies electric power to the control board 16 and various functional components, or the like. The private part washing device 3 functions as a deodorizer having the deodorizing unit 15 as a main unit.

The nozzle unit 12 is disposed at the center of the base 8. The nozzle unit 12 includes a washing nozzle 18 for washing an anus, a nozzle driving mechanism 19 that drives the washing nozzle 18, a washing nozzle 20 for a bidet, which is adjacent to the washing nozzle 18 on right and left sides, the nozzles configuring twin nozzles, and a nozzle driving mechanism 21 that drives the washing nozzle 20.

A pair of right and left washing nozzles 18 and 20 are supported to be extended from and retracted to the base 8 so as to be inclined downward toward the front side, the nozzles being parallel to each other. When not in use, the washing nozzles 18 and 20 are accommodated on an inner side of the shutter 10 when the opening part 9 is closed by the shutter.

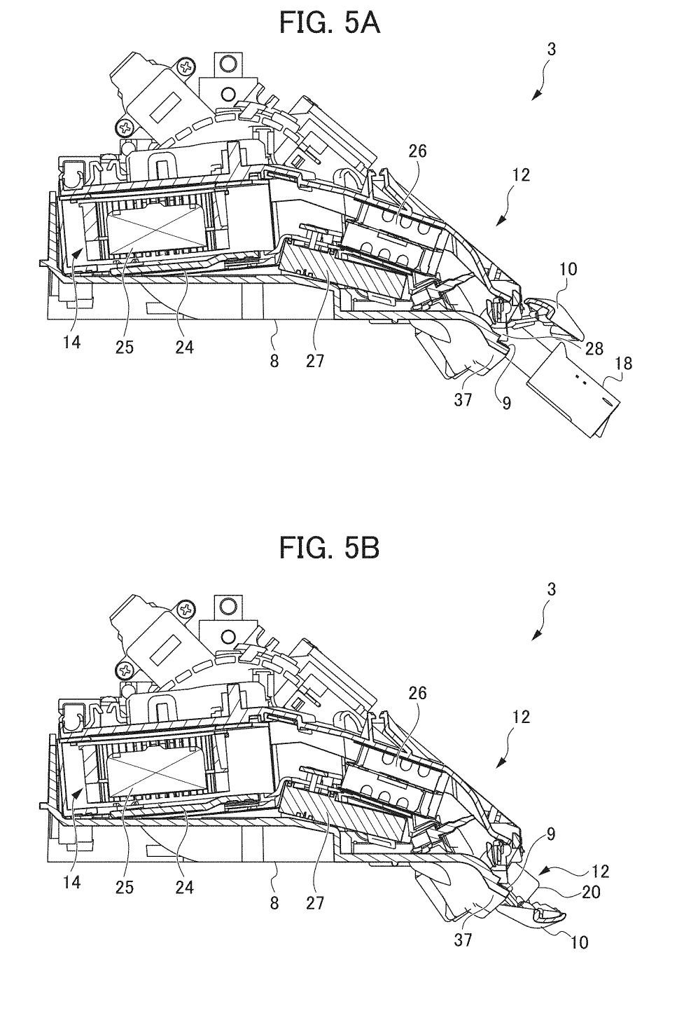

The washing nozzle 18 for washing an anus proceeds obliquely downward by the nozzle driving mechanism 19, and thereby a tip end of the washing nozzle pushes a lower side of an inner surface of the shutter 10 via a protrusion (not illustrated) on the inner surface of the shutter 10. Hence, the shutter 10, which is biased in a direction in which the opening part 9 is closed, is opened while being pivoted upward, and the washing nozzle projects toward a front side of the opening part 9. In this case, the shutter 10 pushed and opened by the washing nozzle 18 comes into a downward opened state of being opened from a lower side of the opening part 9 against a bias force in a direction in which the opening part 9 is closed (refer to FIG. 5A).

The washing nozzle 20 for a bidet proceeds obliquely downward by the nozzle driving mechanism 21, and thereby a tip end of the washing nozzle pushes an upper side of the inner surface of the shutter 10 via a protrusion (not illustrated) on the inner surface of the shutter 10. Hence, the shutter 10, which is biased in the direction in which the opening part 9 is closed, is opened while being pivoted downward, and the washing nozzle projects toward the front side of the opening part 9. In this case, the shutter 10 pushed and opened by the washing nozzle 20 comes into an upward opened state of being opened from an upper side of the opening part 9 against a bias force in a direction in which the opening part 9 is closed (refer to FIG. 5B).

The washing nozzles 18 and 20 have respective discharge holes 22 on tip portions thereof which are positioned on a lower side, the discharge holes 22 discharging washing water supplied by the warm water supply unit 13.

The warm water supply unit 13 is disposed on a right side of the base 8. The warm water supply unit 13 is connected to both the washing nozzles 18 and 20 via individual tube systems, and warm water adjusted to have a preset temperature is supplied as washing water to the washing nozzles 18 and 20.

The air blowing unit 14 is disposed on the right side of the base 8. The air blowing unit 14 includes an air duct 24 that guides external air to the opening part 9, a first blower 25 provided in the air duct 24, and a heater 26 and an ion generator 27 which are provided downstream of the first blower 25 in the air duct 24.

The air duct 24 has an intake port (not illustrated) of external air on an upstream end and a blowout port 28 on a downstream end, the blowout port 28 opening from the opening part 9 to the outside of the case 7. Drive of the first blower 25 causes the air duct 24 to draw external air from the intake port and to blow out airflow from the blowout port 28. The first blower 25 generates an air current from the intake port to the blowout port 28 in the air duct 24. The heater 26 warms air drawn by the air duct 24. The ion generator 27 ionizes the air drawn by the air duct 24 so as to generate plus ions and minus ions.

The air blowing unit 14 drives the first blower 25 and the heater 26, thereby, blowing out hot air from the blowout port 28. In addition, the air blowing unit 14 drives the first blower 25 and the ion generator 27, thereby, blowing out airflow containing plus ions and minus ions from the blowout port 28, and the ions inactivate floating bacteria. Consequently, breeding of the floating bacteria is limited or the bacteria are removed.

The deodorizing unit 15 is disposed on a left side of the base 8. The deodorizing unit 15 includes a deodorizing duct 29 that guides air in the toilet bowl 4 to the outside of the toilet bowl 4, a second blower 30 provided in the deodorizing duct 29, and a deodorizing cartridge 31 provided downstream of the second blower 30 in the deodorizing duct 29.

The deodorizing duct 29 has a suction port 32 on an upstream end, the suction port being formed close to the center on a front side of the base 8, and has a blowout port 33 on a downstream end, the blowout port being formed on the left side of the base 8. Drive of the second blower 30 causes the deodorizing duct 29 to suction the air in the toilet bowl 4 from the suction port 32 and to blow 33 out airflow from the blowout port 33. That is, the deodorizing unit 15 of the embodiment is a non-circulation type (one-pass type) deodorizer. The second blower 30 generates an air current from the suction port 32 to the blowout port 33 in the deodorizing duct 29. The deodorizing cartridge 31 contains a deodorizing catalyst inside, and the air suctioned to the deodorizing duct 29 is caused to pass through the deodorizing cartridge. In this manner, odor contained in the air passing therethrough is removed.

Incidentally, the suction port 32 is formed on a bottom surface of the base 8 and is opened downward toward an inside of the toilet bowl 4. That is, the shutter 10 does not have a suction port inside, and the suction port is formed on the bottom surface of the base 8. Hence, even when the shutter 10 is closed, it is possible to suction air (odor) in the toilet bowl 4.

Incidentally, in the embodiment, an adsorptive deodorant containing activated carbon or the like as a catalyst is employed as the deodorizing cartridge 31; however, a decomposition type deodorant such as a photocatalyst, which has a function of decomposing a substance, by being irradiated with a light ray such as an ultraviolet ray.

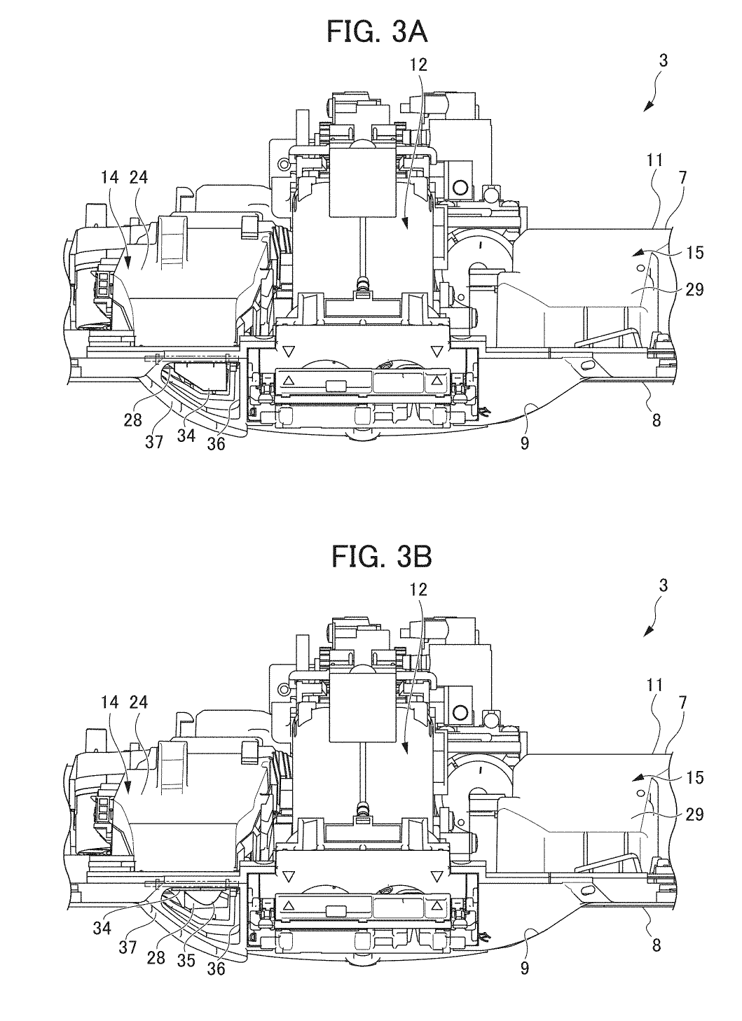

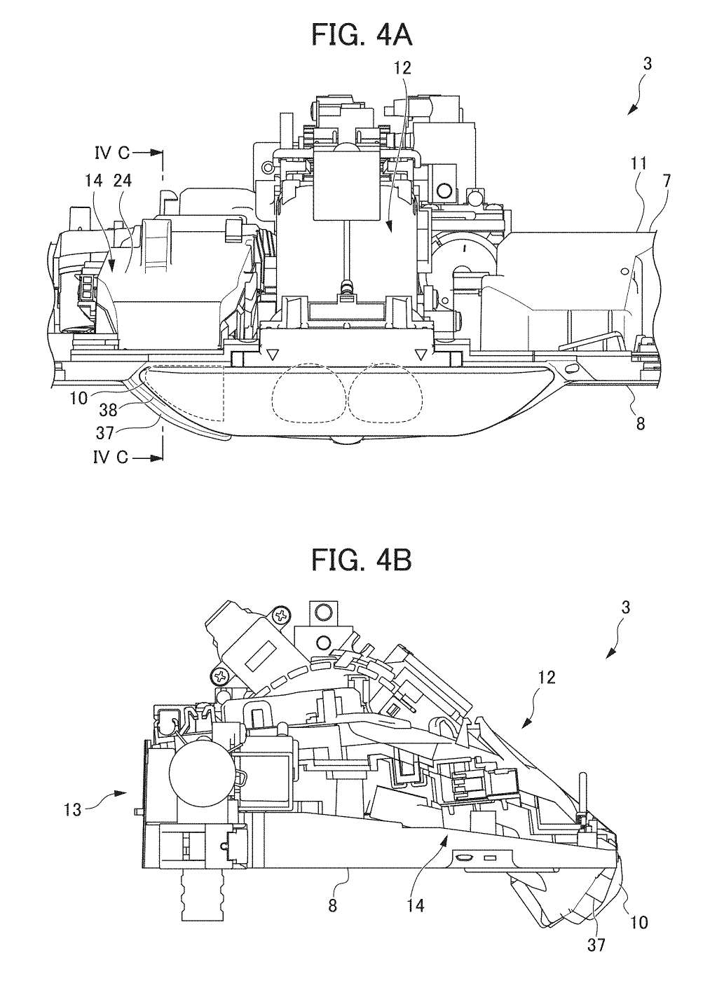

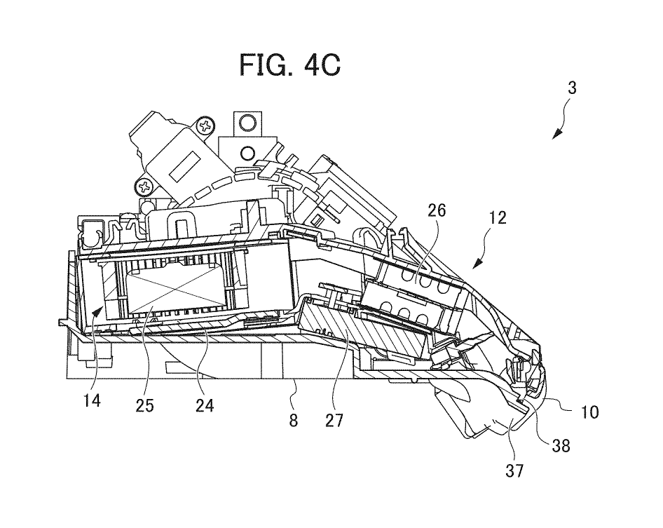

Next, with reference to FIGS. 3A to 5B, a structure of the shutter 10 and a peripheral portion thereof will be described. Here, FIG. 3A is a front view illustrating the opening part 9 and a peripheral portion thereof when the shutter 10 is detached and a second shutter 34 is closed. FIG. 3B is a front view illustrating the opening part 9 and the peripheral portion thereof when the shutter 10 is detached and the second shutter 34 is opened. FIG. 4A is a front view illustrating the shutter 10 in a closing state and the peripheral portion thereof. FIG. 4B is a side view illustrating the shutter 10 in the closing state and the peripheral portion thereof. FIG. 4C is a cross-sectional view of the shutter 10 and the peripheral portion thereof when viewed in a direction of arrow IVC-IVC in FIG. 4A. FIG. 5A is a cross-sectional view of the shutter 10 in a downward opened state and the peripheral portion thereof. FIG. 5B is a cross-sectional view of the shutter 10 in an upward opened state and the peripheral portion thereof.

As illustrated in the drawings, the private part washing device 3 includes the second shutter 34 that opens and closes the blowout port 28 of the air duct 24 on the inner side of the shutter 10, a rib 35 provided on an inner surface of the second shutter 34, a blocking plate 36 provided along an edge of the blowout port 28, and a rib 37 provided along an edge of the opening part 9.

The second shutter 34 has an upper end that is pivotably supported on the edge of the blowout port 28, opens the blowout port 28 with blowout airflow, and closes the blowout port 28 by own weight of the second shutter 34. The second shutter 34 opens the blowout port 28 when airflow is blown out from an air outlet 38 when the shutter 10 is closed.

The rib 35 is configured to be integrated with the inner surface of the second shutter 34 so as to be extended obliquely downward from above toward the center of the toilet bowl 4 in the right-left direction when the second shutter 34 is opened. The rib 35 guides the airflow, which is blown out from the blowout port 28, toward the center in the right-left direction when the blowout port 28 is opened by the second shutter 34.

The blocking plate 36 is provided on a left side of the blowout port 28 along the edge of the blowout port 28. The blocking plate 36 functions as a blocking unit that guides airflow blown out from the blowout port 28 to the air outlet 38 formed between the shutter 10 and the edge of the opening part 9 when the opening part 9 is closed by the shutter 10. The blocking plate 36 prevents the airflow from flowing toward an opposite side of the air outlet 38 in the shutter 10, that is, on a more inner side than the edge of the opening part 9. The blocking unit is not limited to the blocking plate 36, and the blocking unit may be configured of a member (for example, an arm of the shutter 10 or the like) other than the blocking plate 36.

The rib 37 is provided to be curved downward from the opening part 9 and downward from the right side of the blowout port 28, the rib being curved along the edge of the opening part 9. The rib 37 guides the airflow blown out from the blowout port 28 from the air outlet 38, which is formed by a gap formed between the shutter 10 and the edge of the opening part 9 as will be described below, toward the center of the toilet bowl 4 from a right side of an inner surface thereof such that the airflow flows along the inner surface of the toilet bowl 4, when the opening part 9 is closed by the shutter 10. More specifically, the airflow is guided to a position having substantially the same height as that of the blowout port 28 on the right side of the inner surface of the toilet bowl 4, and the airflow is further guided toward a lower side than at the center. That is, the airflow is guided toward the lower side as approaching the center from the right side of the inner surface of the toilet bowl 4.

In addition, as illustrated in FIG. 3A, there is a region NA in which the rib 37 is not formed on a left end of the air outlet 38, that is, on a center side of the opening part 9. The airflow is blown out downward even from the region NA so as to be guided into the toilet bowl 4.

In addition, as illustrated in FIG. 4B, a tip portion of the rib 37 is inclined rearward of the toilet bowl 4 as being downward in side view of the main toilet body 2. That is, the airflow blown out from the blowout port 28 is swirled to flow downward from above along the inner surface of the toilet bowl 4 as illustrated by arrows in FIG. 2 and configures an air current that can inhibit odor in the toilet bowl 4 from diffusing outside the toilet bowl 4.

When the opening part 9 is closed by the shutter 10, the air outlet 38 is formed by the gap formed downward from the opening part 9 and downward from the right side of the blowout port 28, the gap being formed between the shutter 10 and the edge of the opening part 9. The air outlet 38 is formed such that the blowout port 28 is hidden in both a front view and a side view. More specifically, the air outlet 38 is concealed by the shutter 10 in a front view, and the air outlet 38 is concealed by the rib 37 in side view. In addition, a lower side of the air outlet 38 is concealed by the rib 37. Consequently, it is possible to inhibit spray from entering the interior from the air outlet 38.

When the opening part 9 is closed, the shutter 10 and the rib 37 guide the airflow blown out from the blowout port 28 toward the center from the right side of the inner surface of the toilet bowl 4 such that the airflow moves along the inner surface of the toilet bowl 4 from the air outlet 38 (refer to FIG. 4). In addition, when the shutter 10 is in a downward opening state, the shutter 10 guides the airflow blown out from the blowout port 28 in a direction in which the airflow is spread downward from the inner surface of the toilet bowl 4 (refer to FIG. 5A). When the shutter is in an upward opening state, the shutter guides the airflow, which is blown out from the blowout port 28, in a direction toward buttocks (refer to FIG. 5B).

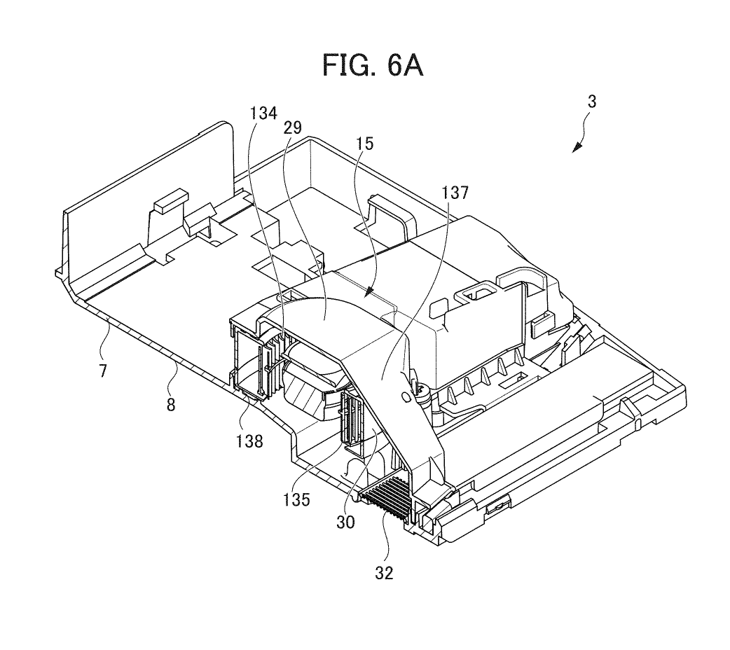

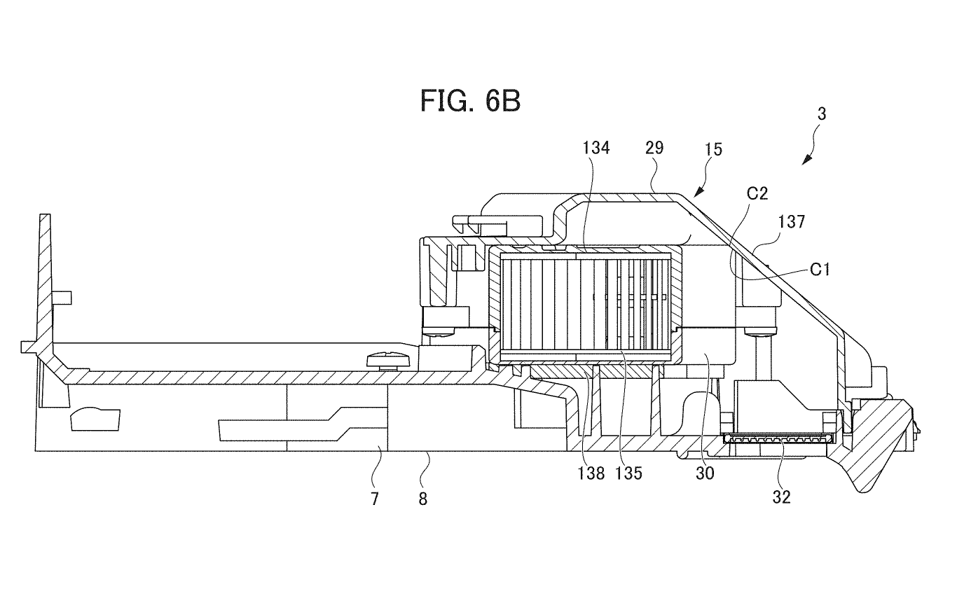

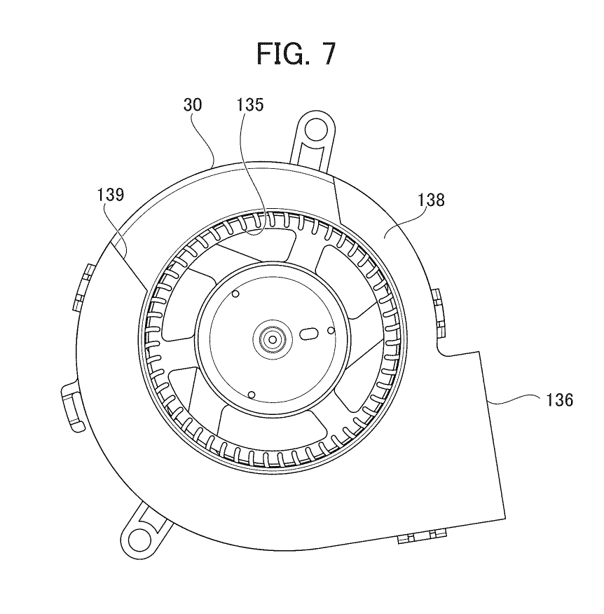

Next, the deodorizing unit 15 will be described with reference to FIGS. 6A to 7. FIG. 6A is a cross-sectional perspective view of the deodorizing unit 15 when viewed in a direction of arrow II-II in FIG. 2. FIG. 6B is a cross-sectional view of the deodorizing unit 15 when viewed in a direction of arrow in FIG. 2. FIG. 7 is a bottom view of the second blower 30 when a packing 138 is bonded.

As illustrated in FIGS. 6A to 7, the second blower 30 of the deodorizing unit 15 is a sirocco fan that has inlet ports 134 and 135 on both end portions of the second blower 30 in an axial direction, respectively, and has a blowout port 136 on a side portion thereof in a direction intersecting the axial direction. The second blower 30 is disposed substantially horizontally, with the inlet ports 134 and 135 being horizontally installed upward and downward.

The deodorizing duct 29 that accommodates the second blower 30 configures a case that forms flow channels of air, which is suctioned from the inlet ports 134 and 135, above and below the second blower 30. The deodorizing duct 29 has a top plate 137 above the suction port 32 formed in the base 8, the top plate 137 being extended obliquely upward toward above the second blower 30 from a side of the second blower 30. In addition, a portion of the deodorizing duct 29 bulges upward, the portion being opposite to the upward inlet port 134 of the second blower 30, and thereby a flow channel communicating with the inlet port 134 is formed while an increase of the second blower 30 in size is avoided.

In addition, as illustrated in FIG. 6B, the second blower 30 is disposed such that an upper corner C1 as a part of the second blower 30 comes into touch with an inner surface of the top plate 137. That is, the top plate 137 has a contact portion C2 that comes into contact with the upper corner C1 of the second blower 30. In addition, the contact portion C2 is notched to form a recessed portion. Consequently, the flow channel communicating with the inlet port 134 is secured, and the second blower 30 is fixed in the deodorizing unit 15 while a space of an upper portion of the top plate is secured. In this manner, vibration or noise is inhibited from occurring.

The deodorizing unit 15 has the packing 138 that is disposed along an edge of the downward inlet port 135 in the second blower 30, the packing being sandwiched between an inner surface of the deodorizing duct 29 and an underside of the second blower 30. The packing 138 is formed into a substantial C shape and has a notch 139 that configures the flow channel of air that is suctioned from the inlet port 135.

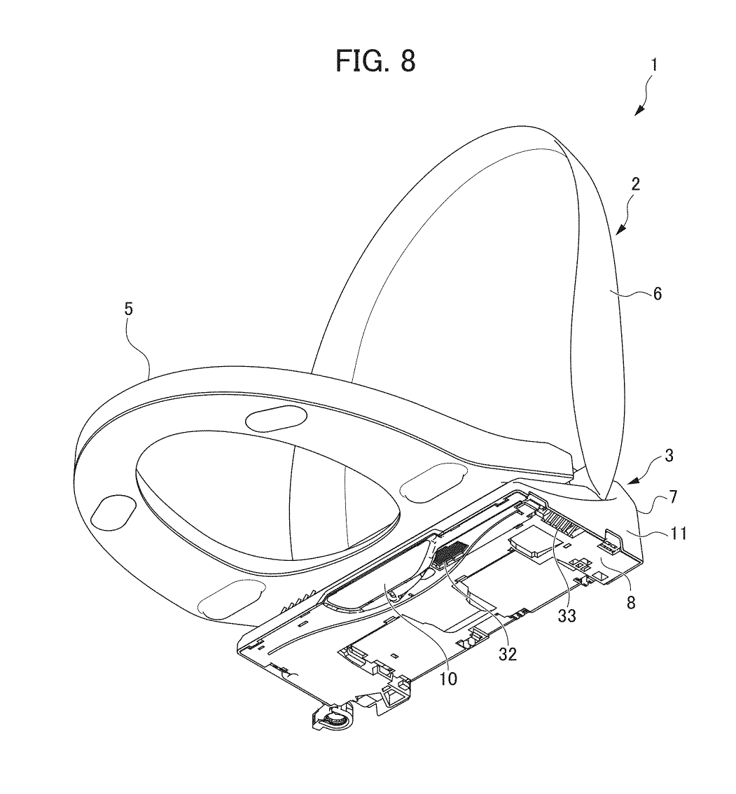

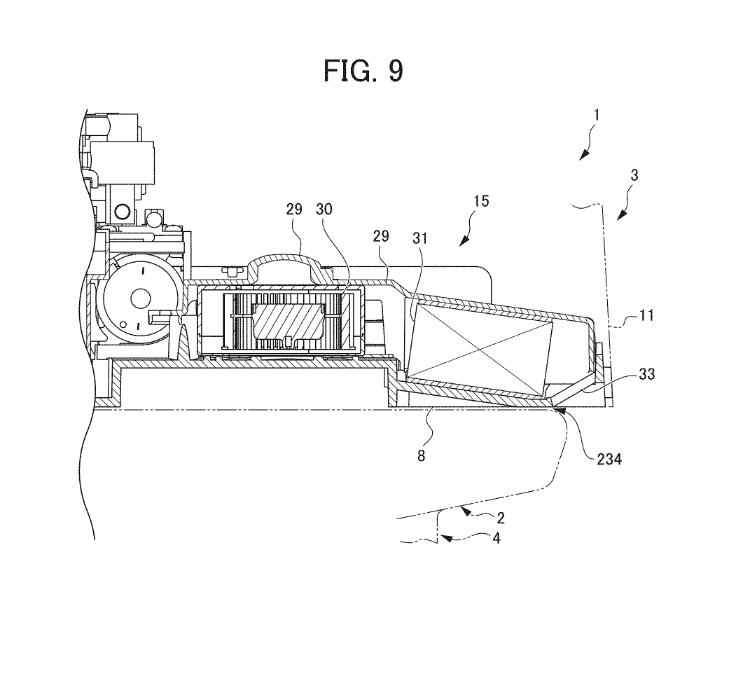

Next, the deodorizing unit 15 will be described with reference to FIGS. 8 and 9. FIG. 8 is a perspective view of the private part washing device 3 when viewed from obliquely below on a front side from the private part washing device 3. FIG. 9 is a cross-sectional view of the deodorizing unit 15 when viewed in a direction of arrow Iv-Iv in FIG. 2.

As illustrated in FIGS. 8 and 9, the blowout port 33 formed on the left side of the base 8 is opened obliquely downward toward a left side. The base 8 is mounted with a gap 234 formed between the blowout port 33 and an upper peripheral edge of the toilet bowl 4. The cover 11 covers the base 8 so as to form a space on the base 8. The cover 11 blocks the blowout port 33 in a left side view.

Incidentally, the blowout port 33 is formed into a grid shape with a plurality of linear holes extended in an inclined direction of the blowout port 33. Here, the blowout port 33 has the grid shape, and thereby an opening area usually decreases; however, the blowout port 33 of the embodiment is inclined, and thus, a sufficient opening area is to be obtained.

The base 8 has a portion in which the deodorizing cartridge 31 is disposed, and the portion is inclined downward toward a direction of the blowout port 33. The deodorizing cartridge 31 is disposed to be inclined downward toward the direction of the blowout port 33. Consequently, the deodorizing cartridge 31 releases air, which has passed therethrough, obliquely downward toward the blowout port 33. As a result, the blowout port 33 smoothly blows out the air, which is released from the deodorizing cartridge 31, to the outer side of the base 8.

Next, an operation mode of the private part washing device 3 will be described.

The private part washing device 3 operates in various operation modes such as a deodorizing mode, a buttock washing mode, a bidet mode, a hot air drying mode, or an intra-bowl bacteria eliminating mode.

The deodorizing mode is an operation mode of deodorizing the inside of the toilet bowl 4. The deodorizing mode includes a first deodorizing mode that is activated when the shutter 10 is closed and a second deodorizing mode that is activated when the shutter 10 is pivoted upward to come into a downward opened state.

The private part washing device 3 in the first deodorizing mode drives the first blower 25 of the air blowing unit 14 after the shutter 10 comes into the closed state such that the airflow blown out from the blowout port 28 is guided from the air outlet 38 of the shutter 10 toward the center of the toilet bowl 4 from the left side of the inner surface thereof, and the private part washing device drives the second blower 30 of the deodorizing unit 15 such that the air in the toilet bowl 4 is suctioned from the suction port 32 and odor is removed in the deodorizing cartridge 31. That is, the airflow blown out from the air outlet 38 flows downward from above along the inner surface of the toilet bowl 4 and is swirled in the toilet bowl 4 so as to be suctioned by the suction port 32 of the deodorizing unit 15. Consequently, while diffusion of the odor upward from the inside of the toilet bowl 4 is suppressed, the odor in the toilet bowl 4 is removed through deodorization. Incidentally, the private part washing device 3 in the deodorizing mode does not drive the heater 26 and the ion generator 27 of the air blowing unit 14.

The private part washing device 3 in the second deodorizing mode causes the washing nozzle 18 for washing an anus to proceed obliquely downward, and thereby the shutter 10 is pivoted upward to come into the downward opened state. Then, the private part washing device 3 drives the first blower 25 of the air blowing unit 14 such that the air blown out from the blowout port 28 is guided in a direction in which the airflow is spread on the inner surface of the toilet bowl 4(refer to the arrows in FIG. 2), and the private part washing device 3 drives the second blower 30 of the deodorizing unit 15 such that the air in the toilet bowl 4 is suctioned from the suction port 32 and odor is removed in the deodorizing cartridge 31. Incidentally, the private part washing device 3 in the deodorizing mode does not drive the heater 26 and the ion generator 27 of the air blowing unit 14.

Incidentally, in the first deodorizing mode and the second deodorizing mode, an output of the second blower 30 on a side of the deodorizing unit 15 is controlled to be higher than an output of the first blower 25 on a side of the air blowing unit 14. Conventionally, in a circulation type deodorizer including one blower, it is necessary to increase an output of the blower so as to make a strong air current in a toilet bowl. However, when the one-pass type deodorizer is used as in the embodiment, the output of the first blower 25 decreases, that is, even when the airflow flowing in the toilet bowl 4 is not strong, the output of the second blower 30 increases, and thereby the odor in the toilet bowl 4 is efficiently suctioned. Consequently, whereas it is difficult for a user to recognize the airflow in the toilet bowl 4, high deodorizing performance is ensured.

The buttock washing mode is an operation mode of washing an anus. The private part washing device 3 in the buttock washing mode causes the washing nozzle 18 for washing an anus to proceed obliquely downward, and thereby the shutter 10 is pivoted upward to come into the downward opened state. Then, the private part washing device 3 drives the warm water supply unit 13 such that the washing water is discharged toward the anus from the discharge hole 22 of the washing nozzle 18, and the private part washing device 3 drives the second blower 30 of the deodorizing unit 15 such that the air in the toilet bowl 4 is suctioned from the suction port 32 and odor is removed in the deodorizing cartridge 31. Incidentally, the private part washing device 3 in the buttock washing mode does not drive the air blowing unit 14.

The bidet mode is an operation mode of washing a private part of a woman. The private part washing device 3 in the bidet mode causes the washing nozzle 18 for washing an anus to slightly proceed obliquely downward, and thereby, the shutter 10 is slightly pivoted upward. Then, the private part washing device causes the washing nozzle 20 for bidet to proceed obliquely downward, and subsequently the shutter 10 is pivoted upward to come into the downward opened state. Then, the private part washing device 3 drives the warm water supply unit 13 such that the washing water is discharged toward a private part of a woman from the discharge hole 23 of the washing nozzle 20, and the private part washing device drives the second blower 30 of the deodorizing unit 15 such that the air in the toilet bowl 4 is suctioned from the suction port 32 and odor is removed in the deodorizing cartridge 31. Incidentally, the private part washing device 3 in the bidet mode does not drive the air blowing unit 14.

The hot air drying mode is an operation mode of drying buttocks with hot air. The private part washing device 3 in the hot air drying mode causes the washing nozzle 20 for bidet to proceed obliquely downward, and thereby the shutter 10 is pivoted downward to come into the upward opened state. Then, the private part washing device 3 drives the first blower 25 and the heater 26 of the air blowing unit 14 such that hot air blown out from the blowout port 28 is guided in a direction toward the buttocks. Incidentally, the private part washing device 3 in the hot air drying mode does not drive the ion generator 27 of the air blowing unit 14 and the deodorizing unit 15.

The intra-bowl sterilizing mode is an operation mode of eliminating bacteria in the toilet bowl 4. The private part washing device 3 in the intra-bowl bacteria eliminating mode causes the washing nozzle 18 for washing an anus to proceed obliquely downward, and thereby the shutter 10 is pivoted upward to come into the downward opened state. Then, the private part washing device 3 drives the first blower 25 and the ion generator 27 of the air blowing unit 14 such that airflow containing plus ions and minus ions which is blown out from the blowout port 28 is guided in a direction in which the airflow is spread downward from the inner surface of the toilet bowl 4. Incidentally, the private part washing device 3 in the intra-bowl bacteria eliminating mode does not drive the heater 26 of the air blowing unit 14 and the deodorizing unit 15.

According to the embodiment, the following effects are to be achieved. The private part washing device 3 according to the embodiment is provided with: the case 7 provided to the rear part of the toilet bowl 4 provided to the main toilet body 2, the case having the opening part 9 opening onto the toilet bowl 4; the air duct 24 having the blowout port 28 from which airflow is blown out, the blowout port 28 opening from the opening part 9 to the outside of the case 7; and the shutter 10 for opening/closing the opening part 9, the shutter 10 forming the air outlet 38 between the shutter 10 and the case 7 when the opening part 9 is closed. When the opening part 9 is closed by the shutter 10, airflow is guided in the direction (first direction) from the air outlet 38 along the inner surface of the toilet bowl 4, and when the opening part 9 is opened by the shutter 10, airflow is guided in the direction (direction different from the first direction) in which the air is spread downward from the inner surface of the toilet bowl 4 or in the direction (direction different from the first direction) toward buttocks. Consequently, when the opening part 9 opened/closed by the shutter 10 is closed, it is possible to guide airflow from the air outlet 38 formed between the shutter 10 and the edge of the opening part 9, and thus it is possible to prevent or inhibit spray, etc. from entering the interior of the private part washing device 3. For example, it is possible to remove odor in the toilet bowl 4 through the deodorization while the opening part 9 is closed as is even when a user uses the toilet in a standing posture.

In addition, in the embodiment, the rib 37 is provided to be curved downward from the opening part 9 and downward from the right side of the blowout port 28, the rib being curved along the edge of the opening part 9. Consequently, when the opening part 9 is closed by the shutter 10, it is possible to cause airflow formed immediately after being blown out from the blowout port 28 to flow along the inner surface of the toilet bowl 4. That is, since the blown-out airflow flows to be curved along the inner surface of the toilet bowl 4 from the beginning, it is possible to reduce diffusion of airflow that is generated when the airflow collides with the inner surface of the toilet bowl 4, and it is possible to guide the airflow to the deodorizing unit so as to perform deodorization while the airflow is efficiently swirled in the toilet bowl 4 such that odor is suppressed.

In addition, in the embodiment, the tip portion of the rib 37 is inclined rearward of the toilet bowl 4 as being downward in side view of the main toilet body 2. Consequently, the airflow blown out from the blowout port 28 flows downward from above along the inner surface of the toilet bowl 4, and the odor in the toilet bowl 4 is removed through deodorization while diffusion of the odor upward from the inside of the toilet bowl 4 is more reliably suppressed.

In addition, in the embodiment, the device includes the blocking plate 36 that guides airflow from the blowout port 28 to the air outlet 38 when the opening part 9 is closed by the shutter 10. Consequently, it is possible to efficiently guide the airflow toward a side of the air outlet 38, and the effects described above are more reliably achieved.

In addition, in the embodiment, the device further includes the first blower 25 provided in the air duct 24, the deodorizing duct 29 that has the suction port 32 which suctions air in the toilet bowl 4, that deodorizes the air in the toilet bowl 4, the air being suctioned from the suction port 32, and that guides the air to the outside of the toilet bowl 4, the second blower 30 provided in the deodorizing duct 29, and the control board 16 that controls drive of the first blower 25 and the second blower 30. In this manner, the control board 16 increases the output of the second blower 30 more than the output of the first blower 25 when the opening part 9 is closed by the shutter 10. Conventionally, in the circulation type deodorizer including one blower, it is necessary to increase the output of the blower so as to make a strong air current in a toilet bowl. However, when the one-pass type deodorizer is used as in the embodiment, the output of the first blower 25 decreases, that is, even when the airflow flowing in the toilet bowl 4 is not strong, the output of the second blower 30 increases, and thereby the odor in the toilet bowl 4 can be efficiently suctioned. Consequently, whereas it is difficult for a user to recognize the airflow in the toilet bowl 4, it is possible to ensure the high deodorizing performance.

In addition, according to the embodiment, the following effects are to be achieved. In the embodiment, the device includes the second blower 30, which has the inlet ports 134 and 135 on both end portions of the second blower 30 in the axial direction and the blowout port 136 in a side portion in the direction intersecting the axial direction, and the deodorizing duct 29, which accommodates the second blower 30 and forms the flow channel of air on both sides of the second blower 30 in the axial direction, the air being suctioned from the inlet ports 134 and 135. Consequently, it is possible to more decrease the second blower 30 in size while the deodorizing function (suction amount) is maintained as is, compared to a case where the blower has a flow channel communicating with the inlet port only on one side. Therefore, it is possible to reduce an installation space, and it is possible to decrease the private part washing device 3 in size. In particular, when the second blower 30 is horizontally installed, it is possible to limit a high dimension of the deodorizer.

In addition, in the embodiment, the deodorizing duct 29 has the top plate 137 that is extended obliquely upward from a side of the second blower 30 to above the second blower 30 and has the portion opposite to the upward inlet port 134 of the second blower 30, the portion bulging upward to form the flow channel. Consequently, it is possible to more decrease a portion on the second blower 30 in size in the deodorizing duct 29, compared to a case where a horizontal flat plate is provided in the entire portion above the second blower 30 in the case 7.

In addition, in the embodiment, a part of the second blower 30 comes into contact with the top plate 137. That is, the second blower 30 is disposed such that the upper corner C1 as a part of the second blower comes into touch with the inner surface of the top plate 137. Consequently, the flow channel communicating with the inlet port 134 is secured, the second blower 30 is fixed in the deodorizing unit 15, and it is inhibit vibration or noise from occurring. In addition, it is possible to stabilize the second blower 30 while the number of components is limited.

In addition, in the embodiment, the packing 138 is disposed along the edge of the downward inlet port 135 and is sandwiched between the inner surface of the deodorizing duct 29 and the underside of the second blower 30. The packing 138 has a notch that configures the flow channel communicating with the inlet port 135. Consequently, it is possible to stabilize the second blower 30 while the flow channel to the downward inlet port 135 is secured.

In addition, according to the embodiment, the following effects are to be achieved. The private part washing device 3 according to the embodiment includes the base 8 that is disposed to the rear part of the toilet bowl 4 provided to the main toilet body 2 and has the blowout port 33 on the side, the blowout port being opened obliquely downward, and the cover 11 that covers the base 8 so as to form the space on the base 8. Consequently, even when the blowout port 33 vertically overlaps the upper peripheral edge of the toilet bowl 4, it is possible to blow out air from the blowout port 33 toward the outer side of the base 8, and thus there is no need to decrease the base 8 in size such that the blowout port 33 is positioned on a more outer side than the upper peripheral edge of the toilet bowl 4. Therefore, it is possible to decrease the private part washing device 3 in size.

In addition, since the private part washing device 3 according to the embodiment includes the cover 11 that covers the base 8, and the cover 11 blocks at least a part of the blowout port 33 in side view, it is possible to hide the blowout port 33 and improve a design quality. In addition, since the base 8 is mounted with the gap formed between the blowout port 33 and the upper peripheral edge of the toilet bowl 4, it is possible to use the base 8 regardless of the size of the toilet bowl 4. Further, since the blowout port 33 opened obliquely downward blows, toward the outer side of the base 8, air released obliquely downward from the deodorizing cartridge 31, it is possible to reduce a pressure loss in the blowout port 33.

As described above, the preferred embodiment of the private part washing device 3 of the invention is described; however, the invention is no limited to the embodiment described above and can be appropriately modified.

### EXPLANATION OF REFERENCE NUMERALS

1 TOILET
2 MAIN TOILET BODY
3 PRIVATE PART WASHING DEVICE (DEODORIZER)
4 TOILET BOWL
5 TOILET SEAT
6 TOILET LID
7 CASE
8 BASE
9 OPENING PART
10 SHUTTER
11 COVER
12 NOZZLE UNIT
13 WARM WATER SUPPLY UNIT
14 AIR BLOWING UNIT
15 DEODORIZING UNIT
16 CONTROL BOARD (CONTROL UNIT)
17 POWER SUPPLY BOARD
18, 20 WASHING NOZZLE
19, 21 NOZZLE DRIVING MECHANISM
22, 23 DISCHARGE HOLE
24 AIR DUCT
25 FIRST BLOWER
26 HEATER
27 ION GENERATOR
28 BLOWOUT PORT
29 DEODORIZING DUCT
30 SECOND BLOWER
31 DEODORIZING CARTRIDGE
32 SUCTION PORT
33 BLOWOUT PORT
34 SECOND SHUTTER
35 RIB
36 BLOCKING PLATE (BLOCKING UNIT)
37 RIB
38 AIR OUTLET
100 TANK
134, 135 INLET PORT
136 BLOWOUT PORT
137 TOP PLATE
138 PACKING
139 NOTCH
234 GAP

1. A deodorizer comprising:
a case provided to the rear part of a toilet bowl provided to a main toilet body, the case having an opening part opening onto the toilet bowl;
an air duct having a blowout port from which airflow is blown out, the blowout port opening from the opening part to the outside of the case; and
a shutter for opening/closing the opening part, the shutter forming an air outlet between the shutter and an edge of the opening part when the opening part is closed,
wherein, when the opening part is closed by the shutter, the airflow is guided in a first direction from the air outlet into the toilet bowl, and when the opening part is opened by the shutter, the airflow is guided in a direction different from the first direction.

2. The deodorizer according to claim 1,
wherein the first direction is a direction along an inner surface of the toilet bowl.

3. The deodorizer according to claim 1 or 2,
wherein the case has a rib provided along an edge of the opening part, the rib guiding the airflow in the first direction from the air outlet when the opening part is closed by the shutter.

4. The deodorizer according to claim 3,
wherein the rib is curved along the edge of the opening part.

5. The deodorizer according to claim 3 or 4,
wherein a tip portion of the rib is provided below the opening part and is inclined toward a rear side of the toilet bowl as being downward in side view of the main toilet body.

6. The deodorizer according to any one of claims 1 to 5, further comprising:
a blocking unit that guides the airflow from the blowout port to the air outlet when the opening part is closed by the shutter.

7. The deodorizer according to any one of claims 1 to 6, further comprising:
a first blower provided in the air duct;
a deodorizing duct that has a suction port which suctions air in the toilet bowl, that deodorizes the air in the toilet bowl, the air being suctioned from the suction port, and that guides the air to the outside of the toilet bowl;
a second blower provided in the deodorizing duct; and
a control unit that controls drive of the first blower and the second blower,
wherein the control unit increases an output of the second blower more than an output of the first blower when the opening part is closed by the shutter.

8. A deodorizer comprising:
a second blower having inlet ports on both end portions, respectively, in an axial direction and a blowout port on a side portion in a direction intersecting the axial direction; and
a case that accommodates the second blower and forms a flow channel of air on both sides of the second blower in an axial direction, the air being suctioned from the inlet port.

9. The deodorizer according to claim 8,
wherein the second blower has the inlet ports that are horizontally installed upward and downward, and
wherein the case forms the flow channel above and below the second blower.

10. The deodorizer according to claim 9,
wherein the case has a top plate that is extended obliquely upward from a side of the second blower to above the second blower and has a portion opposite to the inlet port of the second blower, the portion bulging upward to form the flow channel.

11. The deodorizer according to claim 10,
wherein the second blower has a part that comes into contact with the top plate.

12. The deodorizer according to any one of claims 9 to 11, further comprising:
a packing disposed along an edge of the downward inlet port, the packing being sandwiched between an inner surface of the case and an underside of the second blower,
wherein the packing has a notch that functions as the flow channel.

13. A deodorizer comprising:
a base that is disposed to the rear part of the toilet bowl provided to a main toilet body and has a blowout port on a side, the blowout port being opened obliquely downward; and
a cover that covers the base so as to form a space on the base.

14. The deodorizer according to claim 13,
wherein the cover blocks at least a part of the blowout port in side view.

15. The deodorizer according to claim 13 or 14,
wherein the base is mounted on the rear part of the toilet bowl with a gap formed between the blowout port and an upper peripheral edge of the toilet bowl.

16. The deodorizer according to any one of claims 13 to 15, further comprising:
a deodorizing part that removes odor contained in air, which passes through the deodorizing part, and releases the air, which has passed through the deodorizing part, obliquely downward,
wherein the blowout port blows out the air released from the deodorizing part, to the outside of the base.
